(19) United States
(12) Patent Application Publication
(10) Pub. No.: US 2010/0160669 A1
(43) Pub. Date: Jun. 24, 2010

(54) PROCESS FOR PRODUCING ORGANIC CARBOXYLIC ACID ESTERS

(75) Inventors: Yu Chiao Liu, Kaohsiung City (TW); Chin Yi Lee, Chiayi City (TW); Chia Jung Tsai, Kaohsiung City (TW)

Correspondence Address: INTELLECTUAL PROPERTY / TECHNOLOGY LAW, PO BOX 14329, RESEARCH TRIANGLE PARK, NC, 27709, US

(73) Assignee: CHINA PETROCHEMICAL DEVELOPMENT CORPORATION, Taipei (TW)

(21) Appl. No.: 12/372,334

(22) Filed: Feb. 17, 2009

(30) Foreign Application Priority Data
Dec. 24, 2008 (TW) 97150392

(51) Int. Cl. C07C 69/66

(52) U.S. Cl. 560/179

(57) ABSTRACT

The present invention relates to a process for producing organic carboxylic acid esters, in which an amino-ester exchange reaction of an organic carboxylic acid amide with an ester compound, or with an alcohol compound under a CO pressure, is carried out at a specific temperature in the presence of a catalyst and a promoter, so as to produce an organic carboxylic acid ester.

### CROSS-REFERENCE TO RELATED APPLICATION

This application is filed under the provisions of 35 USC 119 claiming the priority of Taiwan Patent Application No. 097150392 filed Dec. 24, 2008. The disclosure of such Taiwan patent application is hereby incorporated herein by reference, in its entirety, for all purposes.

### FIELD OF THE INVENTION

The present invention relates to a process for producing organic carboxylic acid esters by an amino-ester exchange reaction of an organic carboxylic acid amide with an ester compound, or with an alcohol compound under a CO pressure, at a specific temperature in the presence of a catalyst and a promoter, and more particularly, a process for producing α-hydroxyl carboxylic acid esters from α-hydroxyl carboxylic acid amides.

### BACKGROUND TO THE INVENTION

Organic carboxylic acid esters are important raw materials in the petrochemical industry, which can be widely used in the fields such as fibers, synthetic rubbers, industrial paints, medicines, pesticides and organic solvents. Most of the organic carboxylic acid esters are produced from the reaction of organic carboxylic acids with alcohols, but in some specific petrochemical processes, the organic carboxylic acid esters are obtained from the alcoholysis of alkylnitriles. For example, methyl methacrylate is obtained from the alcoholysis and esterification of 2-hydroxyisobutyronitrile, and lactate is obtained from the alcoholysis of 2-hydroxypropionitrile. The catalyst used in the traditional alcoholysis reaction is sulfuric acid, which will produce a large amount of by-product ammonium sulfate during the alcoholysis. In the past, ammonium sulfate can be used as fertilizers; however, due to the industrial development, the current yield of ammonium sulfate has exceeded the quantity demanded. Under the condition that the process cannot be modified, ammonium sulfate has become a troublesome waste problem and also increase the production cost.

In order to solve the problem of large amount of ammonium sulfate produced during the alcoholysis of alkylnitriles, processes for producing organic carboxylic acid esters without simultaneously producing ammonium sulfate were developed, in which alkyl nitrile compounds are hydrolyzed to obtain organic carboxylic acid amide compounds and then an amino-ester exchange between the organic carboxylic acid amides and ester or alcohol compounds are conducted to produce organic carboxylic acid esters. U.S. Pat. No. 4,055,590 discloses a method for making an organic carboxylic acid ester by the reaction of an organic carboxylic acid amide with methanol in the presence of a metal carboxylate compound as the catalyst at an elevated temperature. The drawback thereof is that the reaction temperature is high and the reaction time is as long as up to 6 hours. JP 53-141216 discloses a process for preparing an organic carboxylic acid ester from an organic carboxylic acid amide and an alcohol in the presence of a metal compound and an oxygen- and nitrogen-containing additive having the chelating property as the catalyst under a condition of high temperature and high pressure. This patented process not only has poor conversion efficiency, but also requires high-priced additives. JP 58-55444 discloses a process for preparing an organic carboxylic acid ester from an organic carboxylic acid amide and a formic acid ester with a Group B metal compound and an oxygen- and nitrogen-containing additive having the chelating property used as the catalyst in a reactor of specific alloy-HC under a condition of high temperature and high pressure. Similarly, this patented process not only has poor conversion efficiency, but also requires co-catalysts. U.S. Pat. No. 4,613,684 discloses a process for the preparation of an organic carboxylic acid ester from an organic carboxylic acid amide and a formic acid ester or alcohol compound in the presence of a metal carbonyl compound and tertiary amine compound as the catalyst under a condition of high temperature and high pressure. The catalytic system used by this patented process is highly toxic and is hardly synthesized and is thus high-priced. U.S. Pat. No. 4,973,739 discloses a process for production of an organic carboxylic acid ester from an organic carboxylic acid amide and a formic acid ester in the presence of a solid acid catalyst under a condition of high temperature. U.S. Pat. No. 4,983,757 discloses a process for production of an organic carboxylic acid ester from an organic carboxylic acid amide and a formic acid ester or alcohol in the presence of an alkaline earth metal oxide catalyst under a condition of high temperature and high pressure. U.S. Pat. No. 4,990,651 discloses a process for producing an organic carboxylic acid ester from an organic carboxylic acid amide and a formic acid ester or alcohol in the presence of sodium methylate as the catalyst under a condition of high temperature and high pressure. This patented process has poor activity and requires long time for achieving equilibrium; also, when other esters (for example, ethyl formate) are used to carry out the reaction, methanol has to be replaced with ethanol and sodium methylate has to be replaced with sodium ethylate so as to avoid the production of formate compounds, which will cause trouble in the separation of products. U.S. Pat. No. 5,194,668 discloses a process for production of an organic carboxylic acid ester from an organic carboxylic acid amide and a formic acid ester or alcohol in the presence of an alkali metal hydroxide catalyst under a condition of high temperature and high pressure. In addition to reaction under high pressure, this patented process needs to conduct a dehydration of the reactants before carrying out the reaction; otherwise, there will be compounds such as organic carboxylic acids and organic ammonium carboxylates produced during the reaction. U.S. Pat. No. 6,310,236 discloses a process for preparing an organic carboxylic acid ester from an organic carboxylic acid amide and an alcohol in the presence of a noble metal compound as the catalyst under a condition of high temperature and high pressure. The drawback of this patented process is that the preparation of the catalyst is highly difficult and costly and the reaction must be carried out at a higher temperature.

In view of the drawbacks of the related art, the process for producing organic carboxylic acid esters according to the present invention conducts an amino-ester exchange reaction of, particularly, an organic carboxylic acid amide containing α-hydroxyl groups (such as α-hydroxy-isobutyric acid amide) with an ester, or with an alcohol under a CO atmosphere, at a low temperature and low pressure by using a metal amide or an alkali alcoholate as the catalyst and ionic liquid as the promoter.

### SUMMARY OF THE INVENTION

The main object of the present invention is to provide a process for producing organic carboxylic acid esters from organic carboxylic acid amides under relatively mild reaction conditions.

Another object of the present invention is to provide a process for producing organic carboxylic acid esters, in which neither costly reaction equipment nor expensive catalysts are required, without producing the by-product of ammonium sulfate from the reaction.

According to the present invention, a metal amide or an alkali alcoholate is used as the catalyst for various amino-ester exchange reactions, together with ionic liquid (IL) as the promoter, so that an amino-ester exchange reaction of an organic carboxylic acid amide (particularly, an organic carboxylic acid amide containing α-hydroxyl groups, such as α-hydroxy-isobutyric acid amide) with an ester, or with an alcohol under a CO atmosphere, is carried out to produce an organic carboxylic acid ester.

As the catalyst for the amino-ester exchange reaction, the metal amide has a structure represented by the formula (I):

M(NH₂)ₓ (I)

in which M is a metal ion of Group IA, IIA or B of all valence numbers. For example, the metal amide can be sodium amide (NaNH₂) etc. Also, the alkali alcoholate can be, for example, sodium methoxide (CH₃ONa), sodium ethoxide, sodium n-propoxide, sodium n-butoxide, potassium methoxide or potassium ethoxide.

As the promoter, the ionic liquid is an ionic type compound constituted of a cation and an anion. The cation constituting the ionic liquid type compound is characterized by having a nitrogen-containing heterocyclic structure and is, for example, a five- or six-membered heterocyclic cation having 1 or 2 nitrogen atoms.

Preferably, the cation constituting the ionic liquid has a structure represented by the formula (II):

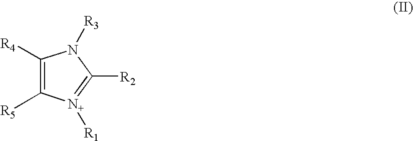

in which R₁ and R₃ are independently selected from the group consisting of C₁₋₁₂ alkyl group, C₁₋₁₂ alkylamino group, C₁₋₁₂ alkoxy group, C₁₋₁₂alkylacyl group, C₃₋₂₀cycloalkyl group, C₃₋₂₀cycloalkoxy group, C₃₋₂₀ cycloalkylacyl group, C₆₋₂₀ aryl group, C₇₋₂₀ arylalkyl group and C₇₋₂₀ alkylaryl group where the C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀cycloalkoxy group, C₃₋₂₀cycloalkylacyl group, C₆₋₂₀ aryl group, C₇₋₂₀ arylalkyl group and C₇₋₂₀ alkylaryl group can further be substituted with halogen, nitro group and/or cyano group; and R₂, R₄ and R₅ are independently selected from the group consisting of hydrogen, halogen, nitro group, cyano group, amino group, C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group where the C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group can further be substituted with halogen, nitro group and/or cyano group.

Alternatively, the cation constituting the ionic liquid has a structure represented by the formula (III):

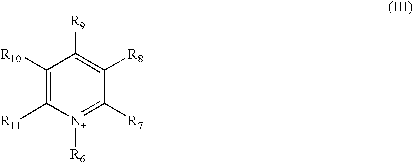

in which R₆ is selected from the group consisting of C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group where the C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group can further be substituted with halogen, nitro group and/or cyano group; R₇, R₈, R₉, R₁₀ and R₁₁ are independently selected from the group consisting of hydrogen, halogen, nitro group, cyano group, amino group, C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group where the C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group can further be substituted with halogen, nitro group and/or cyano group.

In the specification, halogen refers to fluorine, chlorine, bromine or iodine; C₁₋₁₂ alkyl group refers to a straight, branched or cyclic alkyl group having 1 to 12 carbon atoms; C₁₋₁₂ alkylamino group refers to a straight, branched or cyclic alkylamino group having 1 to 12 carbon atoms; C₁₋₁₂ alkoxy group refers to a straight, branched or cyclic alkoxy group having 1 to 12 carbon atoms; C₁₋₁₂ alkylacyl group refers to a straight, branched, or cyclic alkylacyl group having 1 to 12 carbon atoms; C₃₋₂₀ cycloalkyl group refers to a cycloalkyl group having 3 to 20 carbon atoms; C₃₋₂₀ cycloalkoxy group refers to a cycloalkoxy group having 3 to 20 carbon atoms; C₃₋₂₀ cycloalkylacyl group refers to a cycloalkylacyl group having 3 to 20 carbon atoms; C₆₋₂₀ aryl group refers to an aryl group having 6 to 20 carbon atoms; C₇₋₂₀ arylalkyl group refers to an arylalkyl group having 7 to 20 carbon atoms; and C₇₋₂₀ alkylaryl group refers to an alkylaryl group having 7 to 20 carbon atoms.

Specifically, the cation of the promoter can be selected from the group consisting of imidazole compounds, pyrrole compounds, benzimidazole compounds, pyridine compounds, bipyridine compounds, pyridazine compounds, pyrimidine compounds, pyrazine compounds and mixtures thereof.

The example of the anion constituting the ionic type compound includes, but is not limited to, F⁻, Cl⁻, Br⁻, I⁻, PF₆⁻, SbF₆⁻, SCN⁻, HSO₄⁻, CH₃SO₃⁻, CH₃SO₄⁻, AlCl₄⁻, Al₂Cl₇⁻, Al₃Cl₁₀⁻, CH₃CH₂SO₄⁻, BF₄⁻, OH⁻, H₂PO₄⁻, N(CN)₂⁻, CH₃COO⁻, CH₃CO⁻ and (CH₃)₂CO⁻, and is preferably selected from PF₆⁻, HSO₄⁻, BF₄⁻, OH⁻, N(CN)₂⁻ or SbF₆⁻.

The organic carboxylic acid amides used in the present invention are generally represented by R₁CONH₂, in which R₁ is a unsubstituted or substituted alkyl group, or a unsubstituted or substituted aryl group. Specifically, R₁ can be selected from, for example, C₁₋₁₂ alkyl group, C₆₋₁₂ aryl group, or alkyl or aryl group having halogen, nitro group, cyano group, hydroxyl group, alkoxycarbonyl group, acyl group or amino group at the α-site.

The esters used in the present invention refer to low molecular weight organic carboxylic acid esters and are generally represented by R₂COOR₃, in which R₂ and R₃ can be the same or different and each can be substituted or unsubstituted alkyl or aryl group. Specifically, for example, R₂ can be selected from hydrogen, C₁₋₁₂ alkyl group, C₆₋₂₀ aryl group, or alkyl or aryl group substituted with halogen, nitro group, hydroxyl group, alkoxycarbonyl group, acyl group, amino group or cyano group, and R₃ can be selected from C₁₋₁₂ alkyl group, C₆₋₂₀ aryl group, or alkyl or aryl group substituted with halogen, nitro group, hydroxyl group, alkoxycarbonyl group, acyl group, amino group or cyano group.

When the reaction is carried out by using a metal amide as the catalyst, together with an ionic liquid as the promoter, the organic carboxylic acid amide R₁CONH₂ will convert into an ester R₁COOR₃ while R₂COOR₃ will convert into an amide R₂CONH₂ after the reaction is completed. The reaction can be expressed by the following equation:

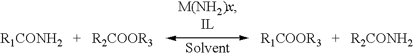

During the reaction, the use of the solvent is to dissolve the catalyst and the promoter. Generally, an alcohol (R₃OH) is used as the solvent; however, the reaction is not limited to using alcohols as the solvent, but can also use other organic solvents having polarity such as acetonitrile, dimethyl sulfoxide, etc. Note that when an alcohol is used as the solvent, the alkyl group R₃ of the alcohol is preferably the same as R₃ of the ester group of the ester to be exchanged to avoid unnecessary side reactions that will increase the difficulty of separation.

The amino-ester exchange reaction can also be carried out by solely using an alcohol R₃OH and the organic carboxylic acid amide compound in the presence of carbon monoxide. The reaction can be expressed by the following equation:

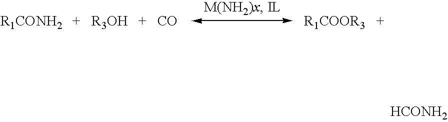

In the solely used alcohol R₃OH, R₃ can be a substituted or unsubstituted alkyl group. Specifically, R₃ can be selected from C₁₋₁₂ alkyl group, or alkyl group substituted with halogen, nitro group, hydroxyl group, alkoxycarbonyl group, acyl group, amino group or cyano group.

According to the present invention, the catalyst is used at an amount of about 1-100%, preferably 10-60%, and most preferably 10-30% of the mole of the organic carboxylic acid amide; the promoter is used at an amount of about 1-100%, preferably 5-60%, more preferably 10-50%, and most preferably 10-40% of the mole of the organic carboxylic acid amide; the ester is used at an amount of 1-100 times, preferably 2-50 times, and most preferably 5-30 times the mole of the organic carboxylic acid amide; and the solvent is used at an amount of 0-15 times the mole of the organic carboxylic acid amide, and the increase in the amount of the solvent will not benefit the productivity. When an alcohol is to be used as the solvent, the alcohol corresponding to the ester group of the ester to be used as the reactant is used in principle; for example, when the reactant is methyl formate or methyl acetate, methanol is used, and when the reactant is ethyl formate or ethyl acetate, ethanol is used, and so on.

According to the present invention, the amino-ester exchange reaction is carried out at a temperature of between 30-200° C., preferably 40-180° C., and more preferably 60-160° C. and at a pressure of between 0-100 kg/cm², preferably 0-60 kg/cm², and more preferably 10-40 kg/cm² for a reaction time of 0.2-5 hours. The reaction is an equilibrium reaction, and the reaction productivity is affected by the kinds and amounts of the organic carboxylic acid amides and esters as used.

### DESCRIPTION OF PREFERRED EMBODIMENTS

The convertibility and selectivity used in the specification are calculated according to the following equations:

Convertibility (%)={[Concentration of Added Organic Carboxylic Acid Amide−Concentration of Unreacted Organic Carboxylic Acid Amide] (mol)/Concentration of Added Organic Carboxylic Acid Amide (mol)}×100%

Selectivity (%)=[Concentration of Organic Carboxylic Acid Ester in the Product (mol)/Concentration of Consumed Organic Carboxylic Acid Amide (mol)]×100%

The catalysts and promoters of the present invention can be applied to various amino-ester exchange reactions of organic carboxylic acid amides. The preferred embodiments below are intended to facilitate understanding of the present invention but not to restrict the practical scope of the present invention.

### Comparative Example 1

8.07 g (0.078 mole) of α-hydroxy-isobutyric acid amide, 23.29 g (0.39 mole) of methyl formate, 0.9087 g (0.0233 mole) of sodium amide and 7.8 g (0.244 mole) of methanol were put into a 130-ml high-pressure reactor of stainless steel with a stirrer. The temperature of the reaction system was elevated to 100° C. with the stirrer started, and the reaction was carried out for 2 hours. After the reaction finished, the reaction solution was cooled down and the product was analyzed with a gas chromatograph. The result is shown in Table 1.

### Example 1

8.07 g (0.078 mole) of α-hydroxy-isobutyric acid amide, 23.29 g (0.39 mole) of methyl formate, 0.9087 g (0.0233 mole) of sodium amide, 1.22 g (0.0078 mole) of 1-butyl-3-methylimidazolium hydroxide ([BMIM]OH) and 6.5 g (0.203 mole) of methanol were put into a 130-ml high-pressure reactor of stainless steel with a stirrer. The temperature of the reaction system was elevated to 100° C. with the stirrer started, and the reaction was carried out for 2 hours. After the reaction finished, the reaction solution was cooled down and the product was analyzed with a gas chromatograph. The result is shown in Table 1.

### Comparative Example 2

8.07 g (0.078 mole) of α-hydroxy-isobutyric acid amide, 0.9087 g (0.0233 mole) of sodium amide and 49.64 g (1.55 mole) of methanol were put into a 130-ml high-pressure reactor of stainless steel with a stirrer, and the reactor was pressurized with carbon monoxide to 30 kg/cm². The temperature of the reaction system was elevated to 100° C. with the stirrer started, and the reaction was carried out for 2 hours. After the reaction finished, the reaction solution was cooled down and the product was analyzed with a gas chromatograph. The result is shown in Table 1.

### Example 2

8.07 g (0.078 mole) of α-hydroxy-isobutyric acid amide, 0.9087 g (0.0233 mole) of sodium amide, 2.2 g (0.0078 mole) of 1-butyl-3-methylimidazolium hexafluorophosphate ([BMIM]PF₆) and 49.64 g (1.55 mole) of methanol were put into a 130-ml high-pressure reactor of stainless steel with a stirrer, and the reactor was pressurized with carbon monoxide to 30 kg/cm². The temperature of the reaction system was elevated to 100° C. with the stirrer started, and the reaction was carried out for 2 hours. After the reaction finished, the reaction solution was cooled down and the product was analyzed with a gas chromatograph. The result is shown in Table 1.

### Comparative Example 3

8.07 g (0.078 mole) of α-hydroxy-isobutyric acid amide, 13.97 g (0.233 mole) of methyl formate, 0.2095 g (0.0038 mole) of sodium methoxide and 7.45 g (0.233 mole) of methanol were put into a 130-ml high-pressure reactor of stainless steel with a stirrer. The temperature of the reaction system was elevated to 100° C. with the stirrer started, and the reaction was carried out for 2 hours. After the reaction finished, the reaction solution was cooled down and the product was analyzed with a gas chromatograph. The result is shown in Table 1.

### Example 3

8.07 g (0.078 mole) of α-hydroxy-isobutyric acid amide, 13.97 g (0.233 mole) of methyl formate, 0.2095 g (0.0038 mole) of sodium methoxide, 1.22 g (0.0078 mole) of 1-butyl-3-methylimidazolium hexafluorophosphate ([BMIM]PF₆) and 7.45 g (0.233 mole) of methanol were put into a 130-ml high-pressure reactor of stainless steel with a stirrer. The temperature of the reaction system was elevated to 100° C. with the stirrer started, and the reaction was carried out for 2 hours. After the reaction finished, the reaction solution was cooled down and the product was analyzed with a gas chromatograph. The result is shown in Table 1.

TABLE 1

| | Convertibility of α-hydroxy- | Selectivity of methyl α- |
|---|---|---|
| | isobutyric acid amide (%) | hydroxyisobutyrate (%) |
| Compar. Ex. 1 | 73.2 | 79.1 |
| Ex. 1 | 74.1 | 90.9 |
| Compar. Ex. 2 | 48.2 | 69.8 |
| Ex. 2 | 48.1 | 81.5 |
| Compar. Ex. 3 | 51.1 | 90.3 |
| Ex. 3 | 47.2 | 98.3 |

The experimental result of Table 1 shows that in the amino-ester exchange reaction of the organic carboxylic acid amide with the ester, or with the alcohol in the presence of carbon monoxide, the use of sodium amide as the catalyst with the ionic liquid added as the promoter can increase the selectivity of the organic carboxylic acid ester of the reaction. In the reaction system of using sodium methoxide as the catalyst, the addition of ionic liquid can also increase the selectivity of the organic carboxylic acid ester.

### Examples 4-6

The procedure of Example 1 was repeated except that the amount of added promoter (the molar percentage of added promoter to the reacted organic carboxylic acid amide) was changed. After the reaction finished, the reaction solution was cooled down and the product was analyzed with a gas chromatograph. The result is shown in Table 2.

TABLE 2

| | | Convertibility of α- | Selectivity of methyl |
|---|---|---|---|
| | Ratio of added | hydroxy-isobutyric | α-hydroxyisobutyrate |
| Example | promoter (%) | acid amide (%) | (%) |
| 1 | 10 | 74.0 | 90.9 |
| 4 | 20 | 72.1 | 91.8 |
| 5 | 30 | 73.1 | 98.9 |
| 6 | 40 | 72.1 | 99.6 |

The experimental result of Table 2 shows that the increase in concentration of the added promoter can increase the selectivity; however, excess promoter may cause the occurrence of the reverse reaction and thus make the convertibility slightly decrease.

### Examples 7-10

The procedure of Example 1 was repeated except that the reaction temperature inside the reactor was changed and 1-butyl-3-methylimidazolium hexafluorophosphate ([BMIM]PF₆) was used as the promoter. After the reaction finished, the reaction solution was cooled down and the product was analyzed with a gas chromatograph. The result is shown in Table 3.

TABLE 3

| | Reaction | Convertibility of α- | Selectivity of methyl |
|---|---|---|---|
| | Temperature | hydroxy-isobutyric | α-hydroxyisobutyrate |
| Example | (° C.) | acid amide (%) | (%) |
| 7 | 80 | 73.1 | 77.4 |
| 8 | 100 | 74.8 | 88.7 |
| 9 | 120 | 62.9 | 95.1 |
| 10 | 140 | 60.7 | 99.5 |

The experimental result of Table 3 shows that the increase in temperature can increase the selectivity of the reaction; however, too high reaction temperature will increase the reaction rate of the reverse reaction and thus make the convertibility of the reaction decrease.

### Examples 11-13

The procedure of Example 1 was repeated except that the CO pressure inside the reactor was changed and 1-butyl-3-methylimidazolium hexafluorophosphate ([BMIM]PF₆) was used as the promoter. After the reaction finished, the reaction solution was cooled down and the product was analyzed with a gas chromatograph. The result is shown in Table 4.

TABLE 4

| | | Convertibility of α- | Selectivity of methyl |
|---|---|---|---|
| | CO pressure | hydroxy-isobutyric | α-hydroxyisobutyrate |
| Example | (kg/cm²) | acid amide (%) | (%) |
| 8 | 0 | 74.8 | 88.7 |
| 11 | 10 | 70.6 | 88.9 |
| 12 | 20 | 71.7 | 95.3 |
| 13 | 30 | 72.7 | 95.3 |

The experimental result of Table 4 shows that the increase in reaction pressure can increase the selectivity of the reaction; however, carbon monoxide may cause the occurrence of the reverse reaction at the same time and thus make the convertibility of the reaction decrease.

### Examples 14-19

The procedure of Example 1 was repeated except that ionic liquids of different anions were used as the promoter. After the reaction finished, the reaction solution was cooled down and the product was analyzed with a gas chromatograph. The result is shown in Table 5.

TABLE 5

| | | Convertibility | |
|---|---|---|---|
| | | of α-hydroxy- | Selectivity of methyl |
| | Kinds of | isobutyric | α-hydroxyisobutyrate |
| Example | promoters | acid amide (%) | (%) |
| 1 | [BMIM]OH | 74.2 | 90.6 |
| 8 | [BMIM]PF₆ | 74.8 | 88.7 |
| 14 | *¹[BMIM]Br | 71.4 | 85.4 |
| 15 | *²[BMIM]HSO₄ | 74.2 | 91.9 |
| 16 | *³[BMIM]N(CN)₂ | 74.8 | 84.9 |
| 17 | *⁴[BMIM]SbF₆ | 76.9 | 89.4 |
| 18 | *⁵[BMIM]BF₄ | 75.3 | 85.1 |
| 19 | *⁶[BMIM]AlCl₄ | 66.5 | 89.7 |

*¹[BMIM]Br represents 1-butyl-3-methylimidazolium bromide.
*²[BMIM]HSO₄ represents 1-butyl-3-methylimidazolium hydrogen sulfate.
*³[BMIM]N(CN)₂ represents 1-butyl-3-methylimidazolium dicyanamide.
*⁴[BMIM]SbF₆ represents 1-butyl-3-methylimidazolium hexafluoroantimonate.
*⁵[BMIM]BF₄ represents 1-butyl-3-methylimidazolium tetrafluoroborate.
*⁶[BMIM]AlCl₄ represents 1-butyl-3-methylimidazolium tetrachloroaluminate.

The experimental result of Table 5 shows that imidazolium salts having different anion structures all can increase the selectivity of the reaction. tetrafluoroborate.

### Examples 20-22

The procedure of Example 1 was repeated except that ionic liquids of different cations were used as the promoter. After the reaction finished, the reaction solution was cooled down and the product was analyzed with a gas chromatograph. The result is shown in Table 6.

TABLE 6

| | | Convertibility | |
|---|---|---|---|
| | | of α-hydroxy- | Selectivity of methyl |
| | Kinds of | isobutyric | α-hydroxyisobutyrate |
| Example | promoters | acid amide (%) | (%) |
| 14 | [BMIM]Br | 71.4 | 85.4 |
| 15 | [BMIM]HSO₄ | 74.2 | 91.9 |
| 16 | [BMIM]N(CN)₂ | 74.8 | 84.9 |
| 20 | *⁷[Pyri]Br | 73.7 | 87.2 |
| 21 | *⁸[EMIM]HSO₄ | 69.6 | 94.7 |
| 22 | *⁹[Pyrro]N(CN)₂ | 72.1 | 92.5 |

*⁷[Pyri]Br represents butylpyridinium bromide.
*⁸[EMIM]HSO₄ represents 1-ethyl-3-methylimidazolium hydrogen sulfate.
*⁹[Pyrro]N(CN)₂ represents 1-butyl-1-methylpyrrolium dicyanamide.

Examples 20, 21 and 22, corresponding to Examples 14, 15 and 16, are the promoters having the structures of the same anions and different cations. The experimental result of Table 6 shows that imidazolium salts having different cation structures all can increase the selectivity of the reaction.

### Examples 23-26

The procedure of Example 1 was repeated except that the ratio of added ester was changed, 1-butyl-3-methylimidazolium hexafluorophosphate ([BMIM]PF₆) was used as the promoter and the reaction was carried out at 120° C. After the reaction finished, the reaction solution was cooled down and the product was analyzed with a gas chromatograph. The result is shown in Table 7.

TABLE 7

| | | Convertibility of | Selectivity of |
|---|---|---|---|
| | Methyl formate/α- | α-hydroxy- | methyl α- |
| | hydroxy-isobutyric | isobutyric acid | hydroxyisobutyrate |
| Example | acid amide | amide (%) | (%) |
| 9 | 5 | 62.9 | 95.1 |
| 23 | 7 | 72.5 | 93.3 |
| 24 | 9 | 78.5 | 96.8 |
| 25 | 15 | 87.7 | 97.9 |
| 26 | 25 | 94.1 | 90.2 |

The experimental result of Table 7 shows that the increase in the amount of methyl formate can increase the convertibility, and can keep the selectivity when used with ionic liquids.

What is claimed is:

1. A process for producing organic carboxylic acid esters, in which an amino-ester exchange reaction of an organic carboxylic acid amide compound with an ester, or with an alcohol under a CO pressure, is carried out at a specific temperature in the presence of a catalyst and a promoter.

2. The process according to claim 1, wherein the catalyst is a metal amide or an alkali alcoholate.

3. The process according to claim 2, wherein the metal amide has a structure represented by the formula (I):
M(NH₂)ₓ (I)
in which M is a metal ion of Group IA, IIA or B of all valence numbers.

4. The process according to claim 2, wherein the alkali alcoholate is selected from the group consisting of sodium methoxide, sodium ethoxide, sodium n-propoxide, sodium n-butoxide, potassium methoxide and potassium ethoxide.

5. The process according to claim 1, wherein the promoter is an ionic liquid, which is an ionic type compound constituted of a cation and an anion.

6. The process according to claim 5, wherein the promoter is a five- or six-membered cyclic compound having 1 or 2 nitrogen atoms.

7. The process according to claim 5, wherein the cation of the promoter is selected from five- or six-membered heterocyclic cations having 1 or 2 nitrogen atoms.

8. The process according to claim 5, wherein the cation of the promoter is selected from the group consisting of imidazole compounds, pyrrole compounds, benzimidazole compounds, pyridine compounds, bipyridine compounds, pyridazine compounds, pyrimidine compounds, pyrazine compounds and mixtures thereof.

9. The process according to claim 5, wherein the cation of the promoter has a structure represented by the formula (II): 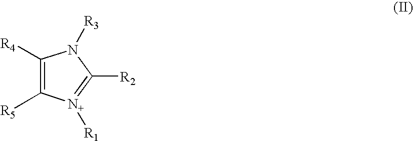
in which R₁ and R₃ are independently selected from the group consisting of C₁₋₁₂ alkyl group, C₁₋₁₂ alkylamino group, C₁₋₁₂ alkoxy group, C₁₋₁₂alkylacyl group, C₃₋₂₀cycloalkyl group, C₃₋₂₀cycloalkoxy group, C₃₋₂₀ cycloalkylacyl group, C₆₋₂₀ aryl group, C₇₋₂₀ arylalkyl group and C₇₋₂₀ alkylaryl group where the C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, aryl group, C₇₋₂₀ arylalkyl group and C₇₋₂₀ alkylaryl group can further be substituted with halogen, nitro group and/or cyano group; and R₂, R₄ and R₅ are independently selected from the group consisting of hydrogen, halogen, nitro group, cyano group, amino group, C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group where the C₁₋₁₂ alkyl group, C₁₋₁₂ alkylamino group, alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group can further be substituted with halogen, nitro group and/or cyano group.

10. The process according to claim 5, wherein the cation of the ionic liquid has a structure represented by the formula (III): 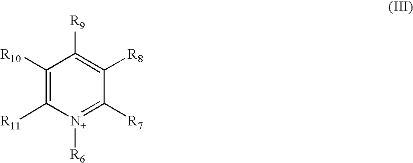
in which R₆ is selected from the group consisting of C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group where the C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group can further be substituted with halogen, nitro group and/or cyano group; R₇, R₈, R₉, R₁₀ and R₁₁ are independently selected from the group consisting of hydrogen, halogen, nitro group, cyano group, amino group, C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group where the C₁₋₁₂ alkyl group, C₁₋₁₂ alkylamino group, C₁₋₁₂ alkoxy group, C₁₋₁₂ alkylacyl group, C₃₋₂₀ cycloalkyl group, C₃₋₂₀ cycloalkoxy group, C₃₋₂₀ cycloalkylacyl group, C₆₋₂₀ aryl group, C₇₋₂₀ arylalkyl group and C₇₋₂₀ alkylaryl group can further be substituted with halogen, nitro group and/or cyano group.

11. The process according to claim 5, wherein the anion of the promoter is selected from the group consisting of F⁻, Cl⁻, Br⁻, I⁻, SCN⁻, HSO₄⁻, CH₃SO₃⁻, CH₃SO₄⁻, AlCl₄⁻, Al₂Cl₇⁻, Al₃Cl₁₀⁻, CH₃CH₂SO₄⁻, OH⁻, BF₄⁻, H₂PO₄⁻, N(CN)₂⁻, PF₆⁻, SbF₆⁻, CH₃COO⁻, CH₃CO⁻ and (CH₃)₂CO⁻.

12. The process according to claim 1, wherein the organic carboxylic acid amide compound has a structure represented by the formula (IV):
R₁CONH₂ (IV)
in which R₁ is selected from the group consisting of C₁₋₁₂ alkyl group, C₆₋₁₂ aryl group, and alkyl or aryl group having halogen, nitro group, cyano group, hydroxyl group, alkoxycarbonyl group, acyl group or amino group at the α-site.

13. The process according to claim 1, wherein the ester has a structure represented by the formula (V):
R₂COOR₃ (V)
in which R₂ and R₃ can be the same or different; and R₂ is selected from the group consisting of hydrogen, C₁₋₁₂ alkyl group, C₆₋₂₀ aryl group, and alkyl or aryl group substituted with halogen, nitro group, hydroxyl group, alkoxycarbonyl group, acyl group, amino group or cyano group, and R₃ is selected from the group consisting of C₁₋₁₂ alkyl group, C₆₋₂₀ aryl group, and alkyl or aryl group substituted with halogen, nitro group, hydroxyl group, alkoxycarbonyl group, acyl group, amino group or cyano group.

14. The process according to claim 1, wherein the alcohol has a structure represented by the formula (VI):
R₃OH (VI)
in which R₃ is selected from the group consisting of C₁₋₁₂ alkyl group, and alkyl group substituted with halogen, nitro group, hydroxyl group, alkoxycarbonyl group, acyl group, amino group or cyano group.

15. The process according to claim 1, wherein the reaction is carried out at a temperature of between 30-200° C.

16. The process according to claim 15, wherein the reaction is carried out at a temperature of between 40-180° C.

17. The process according to claim 16, wherein the reaction is carried out at a temperature of between 60-160° C.

18. The process according to claim 1, wherein the reaction is carried out at a pressure of between 0-100 kg/cm².

19. The process according to claim 18, wherein the reaction is carried out at a pressure of between 0-60 kg/cm².

20. The process according to claim 19, wherein the reaction is carried out at a pressure of between 10-40 kg/cm².

21. The process according to claim 1, wherein the concentration of added promoter is 1-100 mol %, based on the concentration of reacted organic carboxylic acid amide compound.

22. The process according to claim 21, wherein the concentration of added promoter is 5-60 mol %, based on the concentration of reacted organic carboxylic acid amide compound.

23. The process according to claim 22, wherein the concentration of added promoter is 10-40 mol %, based on the concentration of reacted organic carboxylic acid amide compound.

24. The process according to claim 1, wherein the amount of reacted ester is 1-100 times the amount of reacted organic carboxylic acid amide compound.

25. The process according to claim 24, wherein the amount of reacted ester is 2-50 times the amount of reacted organic carboxylic acid amide compound.

26. The process according to claim 25, wherein the amount of reacted ester is 5-30 times the amount of reacted organic carboxylic acid amide compound.
